(19) United States
(12) Patent Application Publication
(10) Pub. No.: US 2004/0225319 A1
(43) Pub. Date: Nov. 11, 2004

(54) Cavity expander

(76) Inventors: Robert Konyn, Gladstone, MI (US); Roger Carviou, Marinette, WI (US)

Correspondence Address: Warn, Burgess & Hoffmann, P.C., P.O. Box 70098, Rochester Hills, MI, 48307, US

(21) Appl. No.: 10/431,262

(22) Filed: May 7, 2003

(51) Int. Cl. A61M 29/00

(52) U.S. Cl. 606/198

(57) ABSTRACT

The present invention relates to an apparatus for use with field dressing an animal. The apparatus is a cavity expander used to spread open the body cavity of an animal after the animal has been field dressed enabling the carcass to cool. The cavity expander is adjustable and includes inner and outer members which are slidably disposed in a telescopic relationship. A detent mechanism, attached to one of the members selectively engages the other member to hold the members in a fixed relationship to achieve a desired overall length of the cavity expander.

### FIELD OF THE INVENTION

The present invention relates generally to an apparatus for use with field dressing an animal and, more specifically, to an apparatus for spreading open the body cavity of an animal after the animal has been field dressed to allow it to cool.

### BACKGROUND OF THE INVENTION

After a game animal is taken, the animal is typically field dressed, which involves opening the body cavity of the animal and removing the internal organs. Once the internal organs are removed, is necessary to hold the body cavity open to enable it to cool.

In the past, once the animal was field dressed, a hunter would often use a stick found on the ground or broken off a nearby tree or bush to prop open the body cavity. Obviously, such a procedure has its drawbacks, specifically finding the right length and type of stick.

Given the inherent drawbacks of using a stick to prop open the body cavity, other types of body cavity spreading devices were developed to prop open the body cavity. These devices are used when field dressing the animal or afterward to keep the body cavity open. Many of these devices are cumbersome, bulky and are often difficult to carry into the field. In addition, several of the prior art carcass spreaders or cavity expanders while adjustable, in that they fit a multitude of animal sizes, are not very compact. Specifically, such adjustable carcass spreaders often require a large support rod upon which the various engagement arms are secured.

Thus, there remains a need in the art for a compact, simple and easy to use carcass spreader for use by a hunter either during or after field dressing an animal.

### SUMMARY OF THE INVENTION

The present invention is a cavity expander for spreading or holding open the body cavity of an animal. The cavity expander includes inner and outer members, wherein the members are slidably disposed in a telescopic relationship. A detent mechanism, attached to one of the members, selectively engages the other member such that the members can be positioned and held in variable positions to achieve a desired overall length of the cavity expander.

### BRIEF DESCRIPTION OF THE DRAWINGS

FIG. 1 is a perspective view of a cavity expander according to the present invention shown engaging the sides of an animal carcass to spread or hold the body cavity open;

FIG. 2 is an exploded perspective view of a cavity expander according to the present invention; and

FIG. 3 is a partial cross-sectional view of the cavity expander according to the present invention illustrating one type of detent mechanism and one type of end cap for use there with.

FIG. 4 is a partial cross-section view of the cavity expander according to an alternative embodiment of the present invention illustrating an additional detent mechanism and end cap suitable for use with the present invention.

FIG. 5 is a partial cross-section view of the cavity expander according to a further alternative embodiment of the present invention illustrating an additional detent mechanism and end cap suitable for use with the present invention.

FIG. 6 is a partial cross-section view of the cavity expander according to an alternative embodiment of the present invention illustrating an additional end cap suitable for use with the present invention.

### DETAILED DESCRIPTION OF THE INVENTION

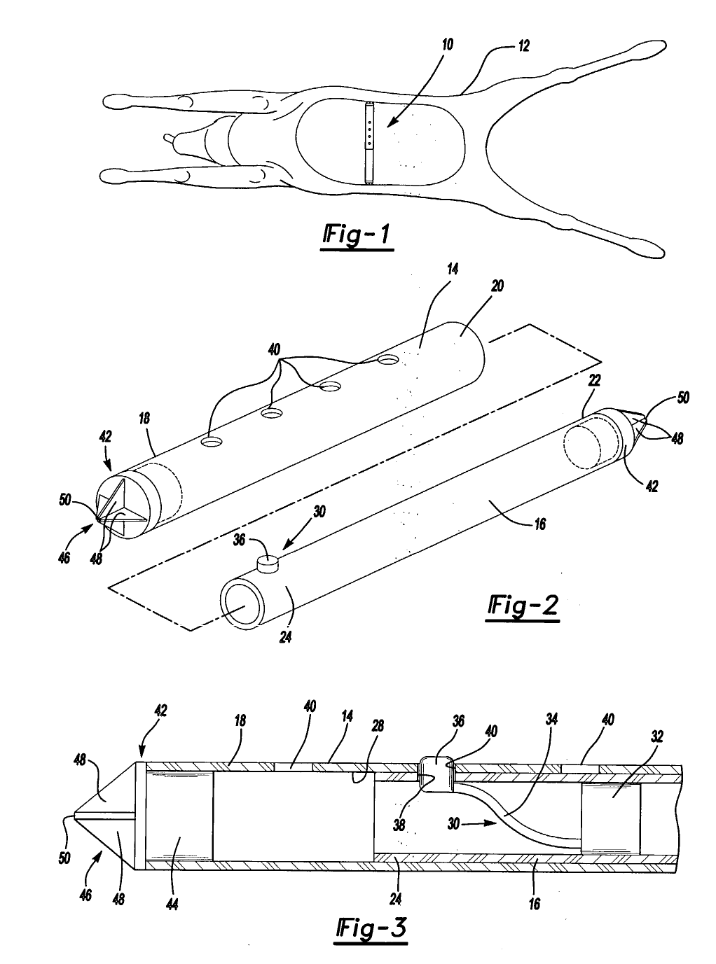

Referring now to FIG. 1 of the drawing, there is shown a cavity expander 10 according to the present invention holding open or expanding the body cavity of an animal carcass 12. The cavity expander 10 includes a first or outer member 14 and a second or inner member 16. As shown in FIG. 2, the second or inner member 16 is sized such that it fits and slides in a reciprocal manner within the first or outer member 14 in a telescopic relationship.

As shown in FIGS. 2-3 both the outer 14 and inner 16 members are cylindrically shaped tubular members wherein the inner member 16 slidably fits within the outer member 14. The first or outer member includes an engagement end 18 and a receptacle end 20. The second or inner member 16 also includes an engagement end 22 and a leading end 24. As illustrated, the leading end 24 of the inner member 16 is inserted into the receptacle end 20 of the outer member 14. Accordingly, as the inner member 16 slides within the outer member 14, the overall length of the cavity expander 10 will vary.

A detent mechanism, seen generally at 30, is used to secure the respective inner 16 and outer 14 members to one another and to maintain the desired overall length of the cavity expander 10. As shown herein, the detent mechanism 30 includes a support member 32 inserted within the leading end 24 of the inner member 16. A spring arm 34 attaches to the support member 32 and has a button 36 located on one end thereof.

It should be understood that the spring arm 34 provides the necessary bias to urge the button 36 outwardly through an aperture 38 in the leading end 24 of the inner member 16. Other mechanisms which operate to urge the button 36 outward through the aperture 38 also come within the scope of the present invention.

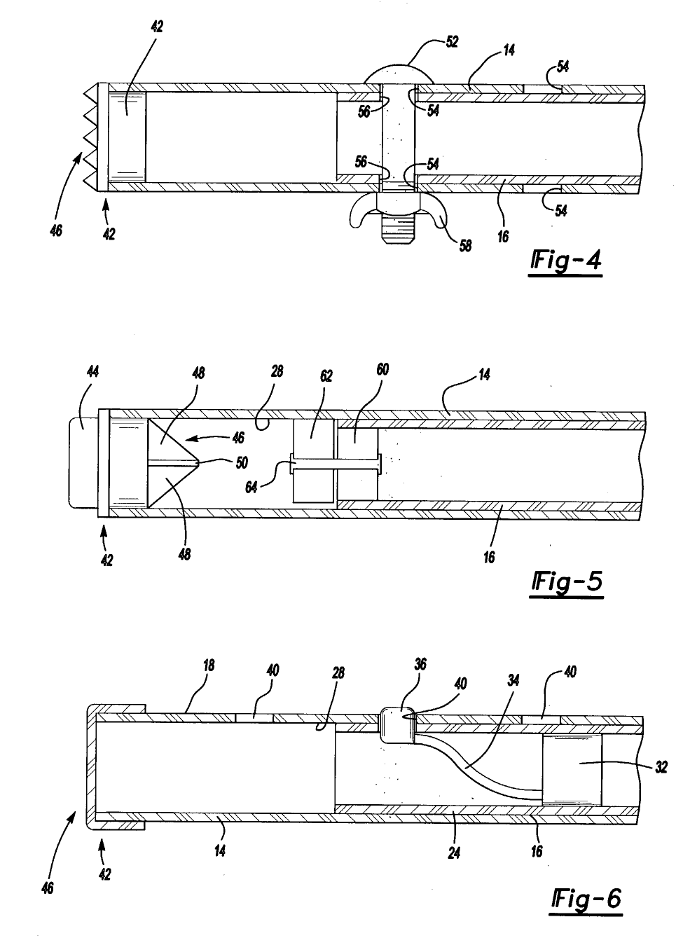

As illustrated in FIG. 2, the outer member 14 includes a plurality of apertures 40 located longitudinally thereon. These apertures 40 form a part of the detent mechanism 30 in that they cooperate with the button 36 to secure the position of the outer member 14 with respect to the inner member 16. To adjust the overall length of the cavity expander 10, the button 30 is depressed or pushed inward until it is below the inner surface 28 of the outer member 14 and thus the outer member 14 is disengaged and uncoupled from the inner member 16 and the inner member 16 is free to move within the outer member 14. Accordingly, the respective outer member 14 and inner members 16 may be repositioned, after which the button 36 is released and allowed to extend outward through the nearest aperture 40 on the outer member 14 to engage and couple the outer and inner members 14, 16. FIGS. 4-5 show further embodiments of detent mechanisms suitable for use with the present invention. FIG. 4 shows a threaded fastener 52 extending through aperatures 54, 56 in the outer 14 and inner 16 members. A nut, such as a wing nut 58, engages the threaded fastener 52 to secure it in place. FIG. 5 shows a twist/cam lock type detent mechanism 30 having a support member 60 and a cam 62 secured for rotation on the support member 60 by a pivot pin 64. As the inner member 16 is axially rotated it causes the cam 62 to rotate and engage the inner surface 28 of the outer member 16 and creates a wedge or binding force which locks the respective outer 14 and inner 16 members in position. It should be understood that any detent mechanism 30 which operates to secure the outer 14 and inner 16 members in position, comes within the scope of the present invention.

As shown in FIGS. 2-3 the cavity expander 10 further includes end caps 42 placed on the respective engagement ends 18, 22 of the outer 14 and inner 16 members. The end caps 42 may take various configurations. As shown in the present embodiment, the end caps 42 included a reduced diameter post portion 44 sized to fit within the engagement ends 18, 22 of the outer 14 and inner 16 members. The end caps 42 further include an engagement portion 46 having a non-slip surface or configuration that contacts the animal carcass 12. The engagement portion 46 shown in FIGS. 2-3, includes a plurality of inclined plate-like members 48 extending outwardly and forming a pointed end 50 which engages the carcass 12 to secure the cavity expander 10 in position.

It should be understood that the end caps 42 may have a variety of engagement portion 46 configurations as long as they operate to prevent the cavity expander 10 from slipping or moving once inserted into the animal carcass 12. As shown in FIG. 4, the engagement portion 46 may have a knurled configuration. Further, in FIGS. 3-5 the end caps 42 are shown inserted within the engagement ends 18, 22 of the outer 14 and inner 16 members, however as shown in FIG. 6 the end caps 42 may have a cup shape and be placed over the respective engagement ends 18, 22 of the respective outer and inner members 14, 16.

As shown in FIG. 5, the end caps 42 may be reversible, in that the engagement portions 46 are inserted within the outer 14 and inner 16 members, prior to use. Wherein the post portion 44 of the end caps 42 extends outward. Prior to use, the engagement portion 46 of the respective end caps 42 is withdrawn from within the respective outer 14 and inner 16 members, the end cap 42 is reversed or rotated, and the post portion 44 is inserted into the engagement end 18, 22 of the outer 14 and inner 16 member. This enables the engagement portion 46 to remain within the cavity expander 10 during transport and prior to use thus reducing the likelihood of it snagging or catching on the hunters clothing or gear during transport. Finally, the end caps 42 can be made of any material suitable to provide a non-slip surface or configuration.

It will thus be seen that the objects of the invention have been fully and effectively accomplished. It will be realized, however, that the foregoing specific embodiments have been shown and described for the purposes of illustrating the functional and structural principles of the invention and is subject to change without departure from such principles. Therefore, this invention includes all modifications encompassed within the scope of the following claim.

What is claimed is:

1. An apparatus for expanding the body cavity of an animal comprising:
a first member;
a second member slidably disposed within said first member in a telescopic relationship; and
a detent mechanism operative to secure said first member and said second member to one another in various positions thus varying the overall length of the apparatus.

2. An apparatus for expanding the body cavity as set forth in claim 1 wherein said the detent mechanism includes a support member connected to the second member;
a spring arm connected to said support member on one end thereof;
a button attached to said spring arm; and
said first member including a plurality of apertures therein wherein the button extends outwardly through one of said apertures in said first member to secure the positional relationship between the first and second members.

3. An apparatus for expanding the body cavity as set forth in claim 1 including said first member having an engagement end and a receptacle end;
said second member having an engagement end and a leading end wherein said leading end of said second member is inserted into said receptacle end of said first member; and
said detent mechanism is secured on said second member adjacent said leading end.

4. An apparatus for expanding a body cavity as set forth in claim 3 including an end cap secured to said engagement end of said first member and an end cap secured to said engagement end of said second member, each of said end caps including an engagement portion.

5. An apparatus for expanding a body cavity as set forth in claim 1 including an end cap secured to one end of said first member and an end cap secured to one end of said second member, each of said end caps including a post portion, a flange portion, and an engagement portion.

6. An apparatus for expanding a body cavity as set forth in claim 5 wherein said engagement portion of said end cap includes a non-slip surface operative to engage said body cavity and hold said apparatus in place.

7. An apparatus for expanding a body cavity as set forth in claim 6 wherein said non-slip surface of said engagement portion includes at least one plate-like member extending outwardly and inclined such that it forms a point which contacts and engages the body cavity.

8. An apparatus for expanding a body cavity as set forth in claim 5 wherein said end cap secured to one end of the first member fits over an outer surface of said first member and said end cap secured to one end of the second member fits over an outer surface of said second member.

9. An apparatus for expanding a body cavity as set forth in claim 5 wherein said end cap is reversible in that the engagement portion includes a post portion which may be inserted into the respective first and second members.

10. An apparatus for expanding a body cavity as set forth in claim 6 wherein said non-slip surface of said engagement portion is a knurled surface.

11. An apparatus for expanding a body cavity as set forth in claim 1 wherein said detent mechanism includes a support member secured to said second member, and a cam member secured to said support member, said cam member operative to engage said first member to secure the positional relationship of said first and second members.

12. An apparatus for expanding the body cavity of an animal comprising:
an outer member having an engagement end and a receptacle end;
an inner member having an engagement end and a leading end, said inner member slidably disposed within said first member in a telescopic relationship wherein said leading end is inserted into said receptacle end;
a detent mechanism operative to secure said outer member and said inner member to one another; and
an end cap placed on each of said engagement ends, said end caps including an engagement portion having a non-slip surface that contacts the body cavity.

13. An apparatus for expanding the body cavity of an animal as set forth in claim 12 wherein said end cap includes a flange portion and a pair of post portions extending outwardly from said flange portion, one of said post portions having said engagement portion thereon.

14. An apparatus for expanding the body cavity of an animal as set forth in claim 13 wherein said non-slip surface of engagement portion includes at least one inclined plate-like member.

15. An apparatus for expanding the body cavity of an animal as set forth in claim 13 wherein said non-slip surface of said engagement portion includes a knurled surface.

16. An apparatus for expanding the body cavity of an animal as set forth in claim 12 wherein said detent mechanism includes a spring biased, said button secured to said inner member and engaging an aperture on said outer member to secure said inner member and outer member in a fixed position.

17. An apparatus for expanding the body cavity of an animal as set forth in claim 12 wherein said the detent mechanism includes a cam lock assembly secured to said inner member, said cam lock assembly including a cam operative to engage said outer member to secure said inner member and said outer member in a fixed position.

18. An apparatus for expanding the body cavity of an animal comprising:
an outer member having an engagement end and a receptacle end;
an inner member having an engagement end and a leading end, said inner member slidably disposed within said first member in a telescopic relationship wherein said leading end is inserted into said receptacle end;
a detent mechanism operative to secure said outer member and said inner member to one another including, a support member secured on said leading end of said inner member, a spring arm having a button located adjacent one end thereof and attached at the other end thereof to said support member, and said leading end of said inner member having an aperature through which said button extends and said outer member having a plurality of apertures wherein said button extends through one of said apertures in said outer member to secure the position of said outer member with respect to said inner member; and
an end cap placed on each of said engagement ends, said end caps including an engagement portion having a non-slip surface that contacts the body cavity.

19. An apparatus for expanding the body cavity of an animal as set forth in claim 18 wherein said non-slip surface of said engagement portion of said end cap includes an inclined plate like member extending outwardly therefrom.

20. An apparatus for expanding the body cavity of an animal as set forth in claim 18 wherein said end cap is removably secured to said engagement end of said inner and outer members.
